(19) United States
(12) Patent Application Publication
(10) Pub. No.: US 2001/0008891 A1
(43) Pub. Date: Jul. 19, 2001

(54) PHARMACEUTICAL COMPOSITIONS COMPRISING A XANTHINE AND A CATECHIN

(76) Inventor: M.T. RAVI SUBBIAH, CINCINNATI, OH (US)

Correspondence Address: FROST BROWN TODD, LLC, 2200 PNC CENTER, 201 E. FIFTH STREET, CINCINNATI, OH, 45202, US

(21) Appl. No.: 09/180,795

(22) PCT Filed: May 15, 1997
(86) PCT No.: PCT/GB97/01335

§ 371 Date: None

(51) Int. Cl. A61K 31/505; A61K 35/78

(52) U.S. Cl. 514/247; 424/725

(57) ABSTRACT

A disintegration machine comprises a casing (1) and at least one element (6, 7, 8) rotatable arranged in the casing for disintegration of material. The casing comprises at least one wall portion (11) extending along the rotatable element. This wall portion is at least partially formed by a device (27) movably arranged in relation to the rest of the casing (1). This device is movable between a first position, in which the device is located in a normal operational position relative to the rotatable element, and a second position, in which the device is moved away from the rotatable element.

This invention relates to pharmaceutical compositions having platelet aggregation inhibitory activity and more particularly to pharmaceutical compositions containing a xanthine such as caffeine and a polyphenol such as a catechin of the type found in Paullinia cupana, Sapindaceae.

Paullinia cupana, commonly known as guarana, is a woody vine or sprawling shrub native to the central Amazon Basin. In the Amazon region, the fruits of guarana are dried under the sun, the seeds are crushed, and aqueous extracts are taken orally. The principal article of commerce of guarana in Brazil is a carbonated soft drink. Guarana is widely used in Brazil as a high caffeine stimulant and in certain local medicines, and has been claimed to have some thinning effects on blood. U.S. Pat. No. 48161594 and a related paper by M. T. R. Subbiah et al, Brazilian J Med Biolo Res (1988) 21:535-538 disclose that an aqueous extract of guarana decreases platelet aggregation in vitro and in vivo. Subbiah et al attempted to isolate the active fractions of guarana. They found that xanthines, mainly caffeine, present in the guarana extract have some anti-aggregatory activity, but the main fraction responsible for the platelet aggregation inhibitory activity was a water-soluble, heat-resistant fraction of unknown composition which appeared to be different from salicylates, nicotinic acid or known xanthines. This fraction not only had platelet aggregation inhibitory activity but also exhibited de-aggregatory action on platelets, a property which was not observed with the xanthines.

Since the Subbiah et al article, a number of papers have been published in which the composition and properties of guarana have been discussed. An article by Houghton in J. Pharm Pharmacol 1991;43(Suppl):125p discusses the caffeine content of guarana and states that one fraction isolated from guarana, when subjected to alkaline hydrolysis, was shown to consist of a complex of caffeine and procyanidin. No mention however is made in this paper about the platelet aggregatory activities of guarana, nor is there any disclosure of any pharmaceutical activities of the proposed complex.

A paper by Houghton et al (J. Pharm Pharmacol, 1992 44:769-771) entitled “Dissolution and Absorption of Caffeine from Guarana” describes investigations into the rate of release of caffeine from capsules of guarana compared to capsules containing an equivalent amount of caffeine. This article also refers to evidence that the caffeine in guarana exists both in the free form and as a complex with tannin, but in the conclusions drawn by Houghton et al from their experiments, they concluded merely that there was no significant difference in the time taken for caffeine to be released from guarana capsules and capsules containing an equivalent amount of caffeine.

A further article by Houghton in the Pharmaceutical Journal, Volume 254, Apr. 1st 1995, pp. 435-436 states that there is evidence that some of the caffeine in guarana exists as a complex with polyphenol but concludes that none of these compounds have been isolated or characterised. Although referring to studies in Brazil which demonstrated that guarana decreases thromboxane synthesis in the blood platelets and also reverses and inhibits platelet aggregation, there is no suggestion that such activity could be due to a complex between caffeine and polyphenols.

Further work by the present inventors on the active fractions of guarana has indicated that the active fractions may contain the polyphenol compound catechin and oligomers and polymers thereof, together with caffeine. Furthermore, it has been discovered that whereas catechins and caffeine on their own are only weakly active as platelet aggregation inhibitors, a mixture of the two demonstrates an activity which is greater than either of the components alone, i.e. a synergistic activity.

Accordingly, in a first aspect, the invention provides a pharmaceutical composition having platelet aggregation inhibitory activity and comprising a mixture of xanthine and a polyphenol; and a pharmaceutically acceptable carrier.

The xanthine and the polyphenol may be present as a mixture of discrete entities, or they may together form a complex.

The polyphenol may be one which upon hydrolysis yields phloroglucinol, ie 1,3,5-trihydroxybenzene. The polyphenol can be one which contains a catechol group, ie an ortho-dihydroxyphenyl group. One such group of compounds is the catechins.

Accordingly, in another aspect, the present invention provides a pharmaceutical composition comprising a xanthine, and a catechin or oligomer or polymer thereof, corresponding substantially to an anti-platelet aggregating catechin compound isolable from Paullinia cupana together with a pharmaceutically acceptable carrier.

In a further aspect, the invention provides a pharmaceutical composition comprising a mixture of xanthine and a catechin or oligomer or polymer thereof, and a pharmaceutically acceptable carrier, the mixture preferably being substantially free of polyphenols other than catechin and/or its oligomers and polymers. By “substantially free” is meant that such other polyphenols, if present at all, are present in the mixture in an amount corresponding to no more than 10% by weight, preferably less than 5% and more preferably less than 2% by weight, relative to the catechin and/or its oligomers and polymers.

In a still further aspect, the invention provides a pharmaceutical composition comprising a mixture having synergistic platelet aggregation inhibitory activity, the mixture consisting essentially of a xanthine and a catechin or oligomers or polymers thereof; together with a pharmaceutically acceptable carrier.

In another aspect, the invention provides the use of a catechin or oligomers or polymers thereof, and a xanthine, for the manufacture of a medicament for the treatment of platelet aggregation mediated conditions.

In addition to platelet aggregation inhibitory activity, compositions of the invention exhibit an ability to deaggregate platelets, and the use of the compositions in the treatment of conditions which are alleviated or prevented by the therapeutic effects of the disaggregation of platelets forms a further aspect of the invention.

Furthermore, the compositions of the invention have activity as inhibitors of thromboxane synthesis, and specifically thromboxane B₂ synthesis. As such they would be expected to be useful in the prevention, treatment or alleviation of disease states and conditions in which thromboxanes play a part.

Thus, in another aspect, the invention provides the use of a combination of a xanthine and a polyphenol as hereinbefore defined, for the manufacture of a medicament for the treatment, alleviation or prevention of diseases or conditions mediated by thromboxanes such as thromboxane B₂.

The xanthines may be of natural or synthetic origin and can be of the formula (I):

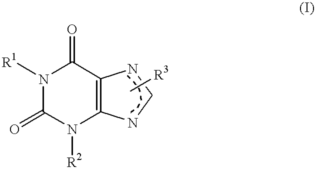

wherein R¹ is H or C₁₋₄ alkyl, R² is C₁₋₄ alkyl and R³ is bonded to either the 7 or 9 positions of the imidazole ring and is hydrogen or C₁₋₄ alkyl.

Preferably R¹ is hydrogen or methyl, R² is methyi and R³ is hydrogen or methyl bonded to the 7-position of the imidazole ring.

Particular examples of xanthines are caffeine (R¹=R²=CH₃, R³=7-CH₃), theobromine (R¹=H, R²=CH₃, R³=7-CH₃) and theophylline (R¹=CH₃, R²=CH₃, R³=7-H). It is preferred that the xanthine is caffeine.

The polyphenols are preferably catechins or oligomers or polymers thereof. The catechins have the general structure:

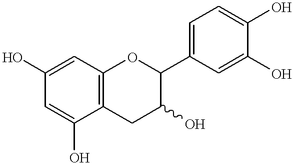

Catechins can exist in a number of optical isomeric and diastereoisomeric forms and all such forms are within the meaning of the term “catechin” as used herein. Thus, for example, the catechins can exist as (+) catechin, (+) epicatechin and (−) epicatechin, the structures for which are as follows:

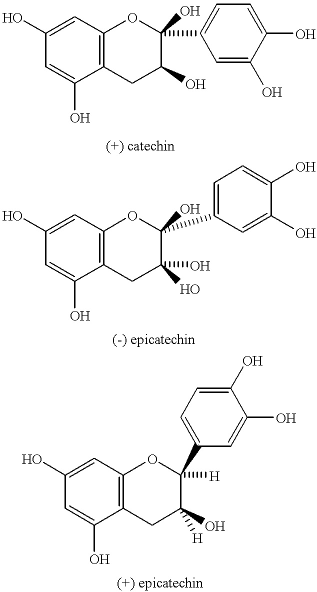

The catechins and their oligomers and polymers can be of synthetic or natural origin. Thus they can be synthesised in accordance with synthetic procedures known in the art. Alternatively, where they are of natural origin, they are conveniently isolated from the Paullinia cupana plant, by solvent extraction and fractionation.

The catechins or oligomers or polymers thereof may be present in the compositions of the invention as a single catechin compound or mixture of catechins in substantially pure form. Alternatively, where they are isolated from P. cupana, there may be present other organic impurities, but it is preferred that the catechins and their oligomers and polymers constitute at least 75% of the plant extract and more preferably at least 90% of the plant extract.

In one embodiment, the invention provides a pharmaceutical composition comprising a pharmaceutically acceptable carrier and a fraction isolable from P. cupana by extraction with ethanol, the fraction being characterised in that it:

(i) has platelet aggregation inhibition activity against ADP-induced platelet aggregation;

(ii) gives a positive colour test with vanillin/sulphuric acid reagent and concentrated hydrochloric acid reagent;

(iii) contains caffeine in complexed or uncomplexed form;

(iv) contains a catechin and/or an oligomer or polymer thereof; and

(v) has an Rf value between about 0.6 and about 0.8 (preferably between about 0.65 and about 0.75) when subjected to thin layer chromatography on 250 micron silica gel G eluted with chloroform: methanol: water (60:40:10 v/v/v).

In the compositions of the invention, it is presently preferred that the xanthine and catechin are present in a molar ratio of between 1:5 and 5:1, typically 1:2 to 2:1 and most preferably 1:1.

It is considered that the xanthine, e.g. caffeine, may form a complex with catechin, or its oligomers or polymers, and indeed caffeine is known to form complexes with a number of compounds, see Reuning et al J. Pharm.Sci. 1968, 57:1355-1341. This Application embraces within its scope formulations in which the caffeine or other xanthine is in either a complexed state or a non-complexed state, or both.

The references to xanthines (e.g. caffeine) and the polyphenols (e.g. catechins) include within their scope salts, particularly pharmaceutically acceptable salts, of these compounds. For example, the xanthines may form acid addition salts with acids such as hydrochloric, sulphuric, acetic, phosphoric, toluenesulphonic, methanesulphonic, benzenesulphonic, malic, maleic, succinic, lactobionic, fumaric and isethionic acids by way of example. The polyphenols useful in the present invention may form salts with bases, for example alkali metal salts such as sodium and potassium salts, or ammonium and substituted ammonium salts. It will be recognised by those skilled in the art that such salts will only be prepared where they are sufficiently stable to be formulated in a pharmaceutical composition and where the process of forming the salt does not bring about decomposition of the parent compound. All such salts, and particularly those which are pharmaceutically acceptable, are within the scope of this invention.

The compositions of the present invention can be administered in standard manner, for example orally, parenterally, transdermally, rectally, via inhalation or via buccal administration, but preferably they are formulated for oral or buccal administration. As such, they can be formulated as solutions, syrups, tablets, capsules, lozenges, inserts and patches by way of example. Such formulations can be prepared in accordance with methods well known per se.

In a particular embodiment, the compositions of the invention can take the form of solid or semi-solid unit dosage forms. For example the compositions can take the form of tablets, granules, lozenges or capsules.

A solid or semisolid dosage form of the present invention can contain, for example, from 50 mg to 500 mg of a mixture of the xanthine and polyphenol (e.g. catechin), more typically 100 mg to 400 mg, and in particular 150 mg to 350 mg, particular unit dosages being approximately 200 mg and 300 mg.

A tablet composition will typically contain one or more pharmaceutically acceptable solid diluents, examples of which include sugars such as sucrose and lactose, and sugar alcohols such as xylitol, sorbitol and mannitol; lactose and sorbitol being particular examples.

The tablets will also typically contain one or more excipients selected from granulating agents, binders, lubricants and disintegrating agents.

Examples of disintegrants include starch and starch derivatives, and other swellable polymers, for example crosslinked polymeric disintegrants such as cross-linked carboxmethylcellulose, crosslinked polyvinylpyrrolidone and starch glycolates.

Examples of lubricants include stearates such as magnesium stearate and stearic acid.

A capsule composition typically will comprise an outer shell or casing which may, for example, be formed from hard or soft forms of gelatine or gelatine-equivalents in conventional fashion. The outer shell is filled with a mixture of a xanthine and a polyphenol (e.g. a catechin) in accordance with the invention. The mixture constituting the capsule filling may be in the form of a powder, or granules, or beads, or may be in the form of a liquid or semi-solid. Where the mixture is in the form of granules, the granules can consist of the xanthine/polyphenol mixture alone, or granulated together with a granulating agent, or they can additionally comprise a solid diluent, for example of the type set forth above. The granules can be wet granulated or dry granulated as desired.

Where the capsule filling is in liquid or semi-solid form, the mixture of xanthines and polyphenols (e.g. catechins) can be dissolved or suspended in a semi-solid carrier material such as a polyethylene glycol or a liquid carrier such as a glycol, e.g. propylene glycol, or glycerol. In general it is preferred that the capsule filling is in solid or semi-solid form when hard gelatine capsules are used; liquid or semi-solid forms being preferred with soft gelatine capsules.

The compositions of the invention have platelet aggregation inhibitory activity and thromboxane synthesis inhibitory activity. As such, the compositions of the invention are useful in the treatment of conditions and disorders in which aggregation of blood platelets play a part and/or which are mediated by thromboxane. Examples of clinical indications in which the compositions of the present invention will be of interest include the treatment or management of post myocardial infarction, coronary thromboses, coronary artery by-pass grafts, cardiac valve replacement and peripheral and vascular grafts. In addition, the compositions of the invention will be of interest in the treatment of migraine.

In a further aspect, the invention provides a method of treatment of a condition or disorder mediated by platelet aggregation, the method comprising administering to a patient in need thereof an effective platelet aggregation inhibiting amount of a composition as hereinbefore defined.

The quantity of drug administered to a patient per day will depend upon the particular condition or disease under treatment and its severity, and ultimately it will be at the discretion of the physician. The amount administered however will typically by a non-toxic amount effective to treat the condition in question.

A typical daily dosage regime for a human patient suffering from a platelet aggregation mediated disease would be from 0.0001 to 0.1, preferably 0.001 to 0.05 gram per kilogram body weight. The compositions can be administered in single or multiple dosage units per day, for example from one to four times daily, preferably one or two times daily.

The compositions of this invention, in addition to containing a xanthine and a polyphenol such as a catechin or oligomer or polymer thereof, can also contain other therapeutic agents selected from antiarrhythmics, ACE inhibitors, beta blockers, vasodilators, other platelet aggregation inhibitors, phosphodiesterase inhibitors, hypolipidaemics by way of example.

The invention will now be illustrated by reference to the following non-limiting examples.

### EXAMPLES

### Example 1

Isolation of a Caffeine-Catechin Mixture from Guarana

Powdered Guarana (Rio Trading Co., Brighton, England) (20 g) was extracted with 95% ethanol (100 ml) and the resulting extract was then filtered through filter-paper. The ethanolic guarana extracts were subjected to thin-layer chromatography (TLC) on 250 μm silica gel G plates (Fischer Chemicals) using chloroform: methanol:water (60:40:10, v/v/v) as the eluting solvent. This system (hereinafter referred to for convenience as the CMW System) was found to be capable of separating guarana tannins. Pure standards for tannic acid, catechin, epicatechin and other tannins were spotted along the side of the samples and were detected after spraying the chromatogram with 10% phosphomolybdate spray (10 grams in 100 ml ethanol). Different bands were scraped and eluted with 95% ethanol, each eluate being centrifuged to remove the gel. The supernatant was dried under nitrogen and the residues were reconstituted either with distilled water (for platelet aggregation studies) or ethanol (for further TLC purification with the same solvent system when needed.

The fractions isolated by TLC were tested for antiplatelet aggregatory activity as described below and were subjected to colour tests using vanillin/sulphuric acid and concentrated hydrochloric acid test reagents. The antiplatelet aggregatory activity was measured using human platelet rich plasma as described below, and in Subbiah et al. Brazilian Journal of Medical and Biological Research 1988; 21: 535-538. The colour reactions were carried out as described in Deshpande et al. Crit. Rev. Nutr. 29, 401-449. The results of the antiplatelet aggregation and colour tests are shown in Table 1 below.

TABLE 1
ANTIPLATELET AGGREGATORY ACTIVITY AND OTHER
PROPERTIES OF GUARANA FRACTIONS AND
STANDARD TANNINS

| | Dis- | | | |
|---|---|---|---|---|
| | tance | | | |
| Compound | migrat- | | | Antiplatelet |
| or | ed | Colour Reactions with | | Aggregatory |
| Fraction | (cm) | Vanillin/H₂SO4 | Con HCl | Activity |
| Tannic Acid | 2.5 | Pink | Brown | |
| Gallic Acid | 5.9 | L. Pink | L. Yellow | |
| Chrvsin | 10.5 | Pink | G. Yellow | |
| Myricetin | 9.0 | Yellow | Yellow | |
| Morin | 8.5 | Red | G. Yellow | |
| Kaemoferol | 9.9 | Yellow | Yellow | |
| Phlorectin | 9.9 | Pink | Yellow | |
| Apigenin | 9.9 | Yellow | G. Yellow | |
| Ellagic Acid | 1.0 | L. Pink | Grey | |
| Catechin | 9.0 | D. Red | Brown | + |
| Epicatechin | 9.0 | D. Red | Brown | + |
| Guarana-1 | 1.0 | No colour | No colour | |
| Guarana-2 | 2.5 | D. Red | Pink | +++ |
| Guarana-3 | 3.0 | Pink | Colourless | |
| Guarana-4 | 4.7 | D. Red | Pink | +++ |
| Guarana-5 | 5.7 | Pink | Colourless | |
| Guarana-6 | 7.2 | D. Red | Pink | |
| Guarana-7 | 8.1 | Yellow | Colourless | |
| Guarana-8 | 9.1 | D. Red | Brownish Red | +++ |

The most active fraction, which migrated 9.1 cm under the conditions and had an Rf value of 0.72, is identified in Table 1 as “Guarana 8” and referred to hereinafter for convenience as Band #8). This fraction was subjected to repeated thin layer chromatography and checked for its effect on platelet aggregation. It was noted that there was considerable loss of activity with re-chromatography.

GC/MS Analysis of the Extracts

The active bands and whole extract were subjected to gas chromatography/mass spectrometry (GC/MS). The instrument used in the analyses of the extracts was a Hewlett Packard 5989A Mass Spectrometer in combination with a 5890 Series II gas chromatograph. The mass spectrometer was operated at 70 ev in the full scan, electron ionization mode. The masses scanned were from 50 to 650 amu. The gas chromatograph utilized a Hewlett Packard ultra-1 capillary column, which is a 24 x×0.2 mm×0.33 μm film, cross-linked methyl silicone column. The injection port temperature was set at 265° C., with the interface at 280° C. and the column temperature was programmed from 80° C., with a hold time of 0.5 minutes, to 295° C. at 15°/minute. The spectral library utilized was NIST version E.00.00.

A preliminary gas chromatography/mass spectrometric analysis of the active fraction (Rf. 0.72) (Band #8) was made after purification of the bands. The two major compounds tentatively identified in the mass spectrum were catechin and its epimer epicatechin. These compounds could only be identified after trimethylsilylation of the samples.

The active fractions of the ethanolic extract were then analysed by GC/MS analysis at different stages of repeated purification and compared to the original guarana extract (GO). When GO was analysed underivatised, only three peaks appeared. The major component (Peak 2) was identified as caffeine. Peaks 1 and 3 were identified as xanthosine and oleic acid, respectively. After GO was trimethylsilylated, a number of peaks appeared on the chromatograph. Peak 1 corresponded to caffeine, and epicatechin and catechin were identified by MS analysis. Peaks 2-7 were analysed by MS and were identified as hexadecanoic acid, oleic acid, octadecanoic acid, eicosanoic acid, and α-glycopyranoside derivatives, respectively. When the active band (GL) was initially purified and separated from the rest of the guarana components, two significant major peaks (catechins) and small amounts of minor peaks (2, 3, 4 fatty acids) remained. Upon further purification (TB-9 and TB-23) the bands essentially contained catechins. It was noted that as the original active band (Band #8) was purified successively it resolved into two major bands (upper and lower) and a minor band (Band C) in thin layer chromatography. When these fractions were checked for platelet aggregation, the lower and upper bands were more active than band C.

HPLC Analysis of the Extracts

In order to ensure that any higher molecular weight compounds or catechin oligomers and polymers not susceptible to detection by mass spectrometry were being detected, further analysis of each of these bands was carried out using HPLC/MS. HPLC/MS was carried out on a Hewlett-Packard HP-1090 Series II liquid chromatograph equipped with variable wavelength detector and on-line recorder using a Supeleosil LC/18 (5 μ) 25.0×4.6 cm diameter reverse phase column. The column was eluted using gradient elution conditions with methanol and 1% acetic acid (20-100% methanol over 30 minutes at a flow rate of 0.5 ml/minute.

The HPLC was interfaced with a HP-API-electrospray detector which was operated in positive or negative mode. With some compounds, the addition of NH₃ was required in order to get better ionization, and this was accomplished with a post column 3-way mixing chamber (as dead volume) which allowed the HPLC element to be mixed with 1% NH₃ in 1-propanol. Additionally LOOP injection (no column) was performed in the testing of samples.

HPLC chromatography of the total extract (TG) of guarana and Band #8 (similar to GL) illustrated that the total extract contained significant amounts of caffeine along with catechin and epicatechin, respectively whilst Band #8 (active band after purification) still contained significant amounts of caffeine along with catechin and epicatechin. The total guarana extract yielded catechins but also material which ionized extensively eluting at 25-30 minutes. Caffeine was not detected in the total extract when tested for using the negative electrospray technique (but see the discussion on the analysis of Band #8 below).

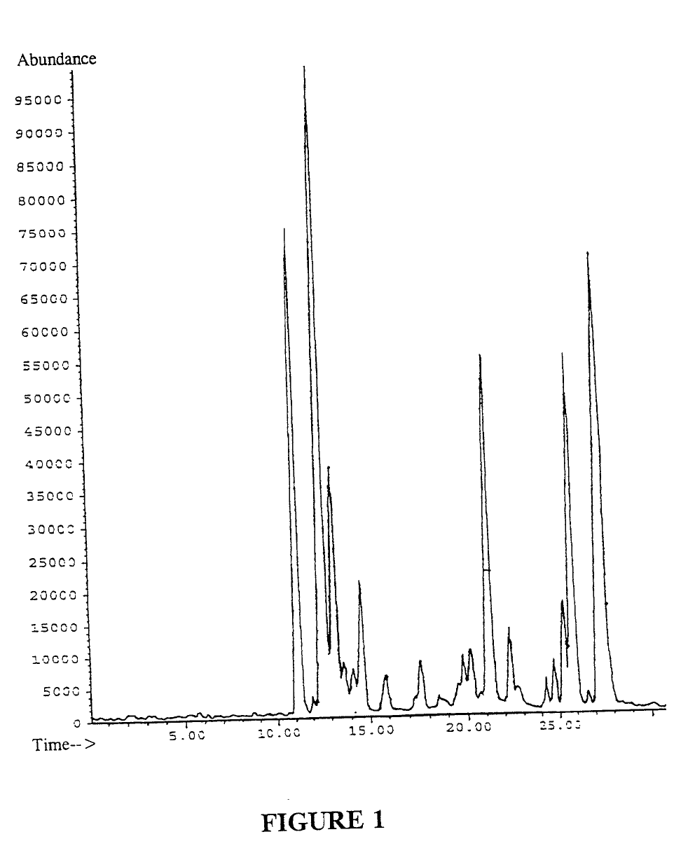

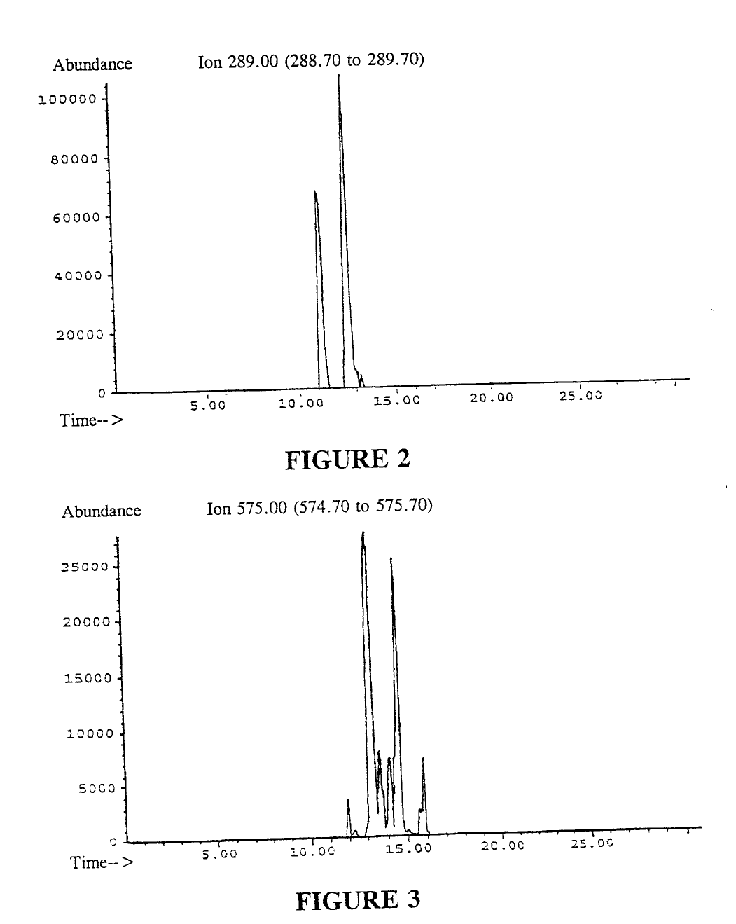

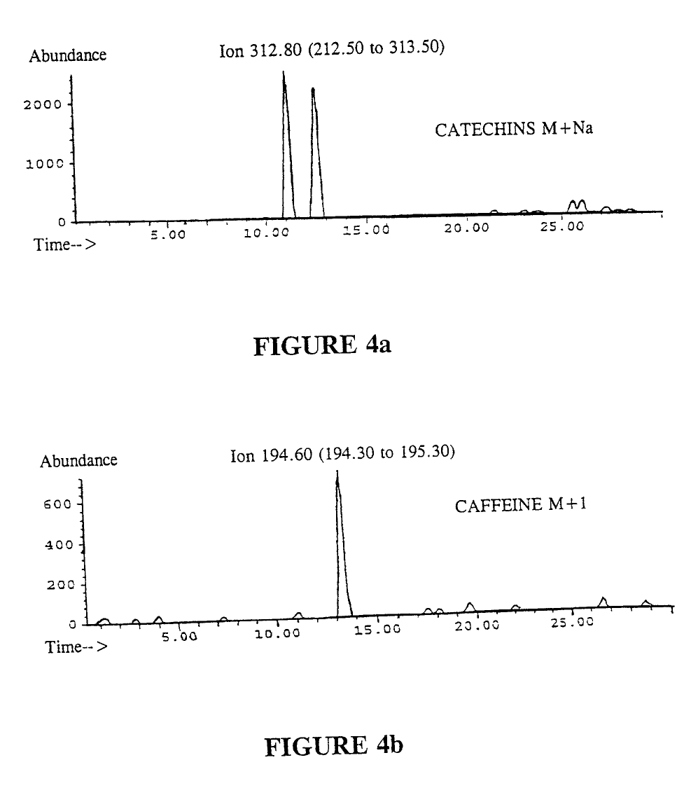

Band #8, when subjected to HPLC using the negative electrospray detection method predominantly yielded ions at m/z 289 (corresponding to catechins), but also yielded ions corresponding to dimers of catechins (ion m/z 575) at 15 minutes retention time; see FIGS. 1 to 3.

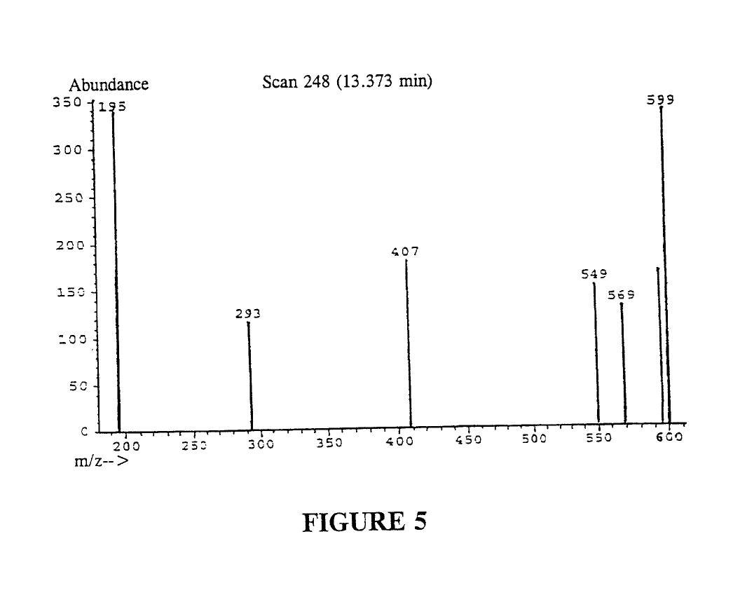

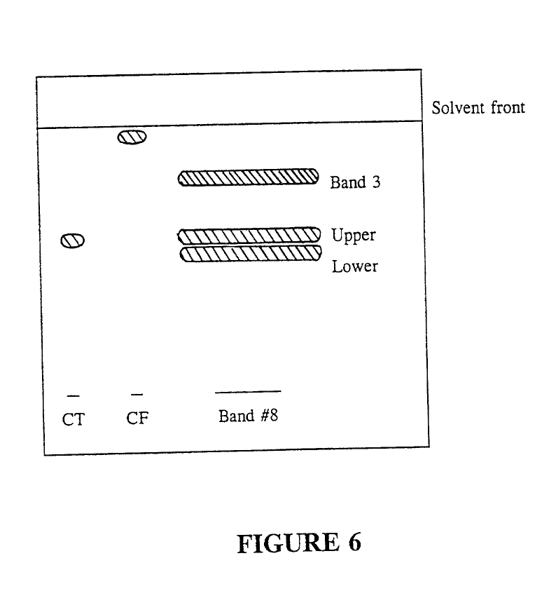

When Band #8 was subjected to HPLC using positive electrospray detection, caffeine was detected, as were sodium salts of catechin—see FIGS. 4a, 4b and 5. However, caffeine was absent from the purified upper and lower bands.

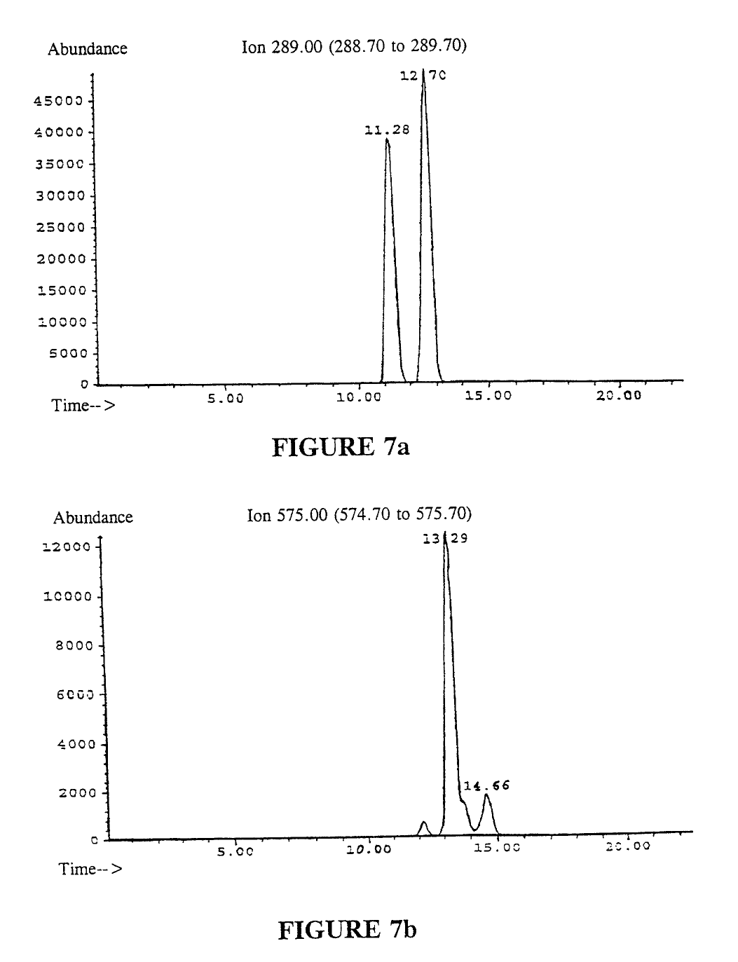

Thin layer chromatography of Band #8 using the chloroform: methanol: water (60:40:10) solvent system on silica gel G yielded three bands (upper, lower, and band C) as shown in FIG. 6. All three bands were only visible soon after removal of the plates from the solvent tank. After spraying with 10% phosphomolybdic acid, the two bands merged into one.

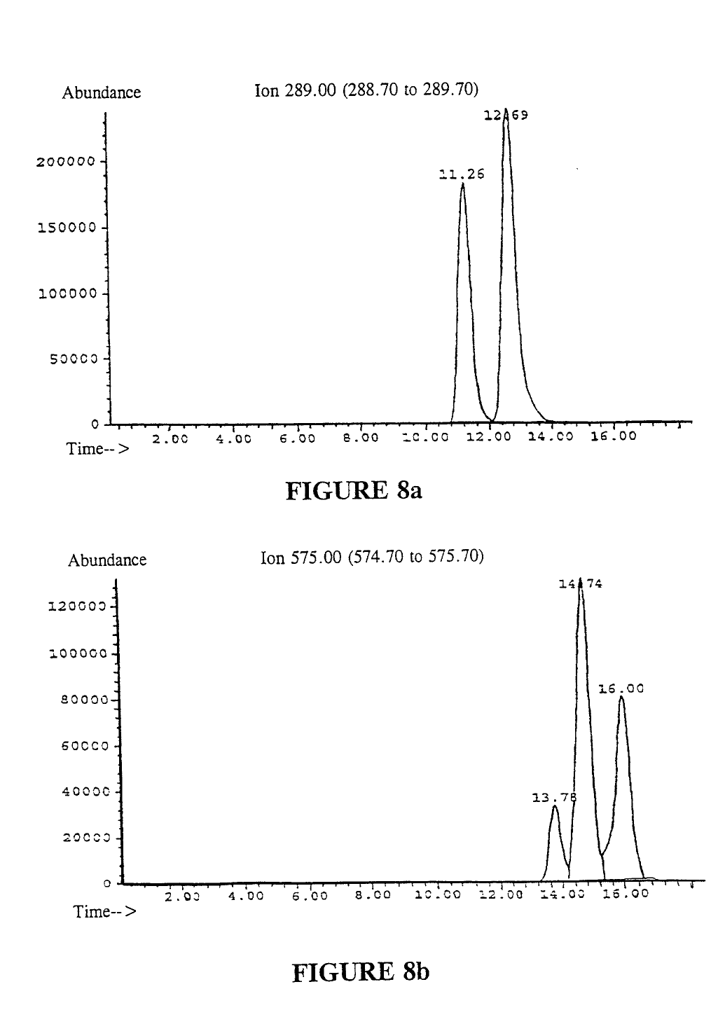

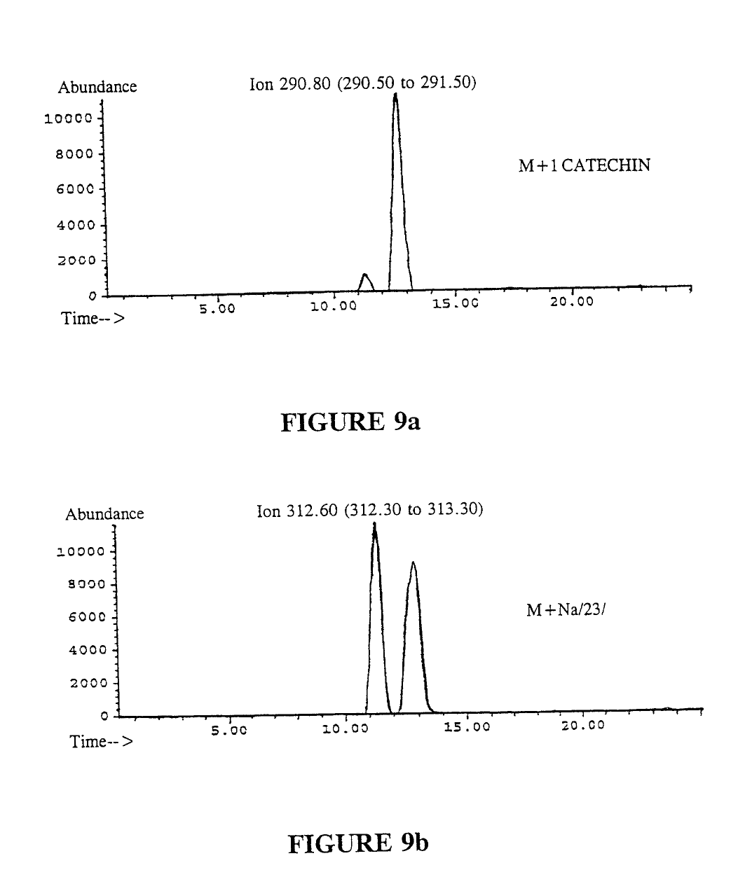

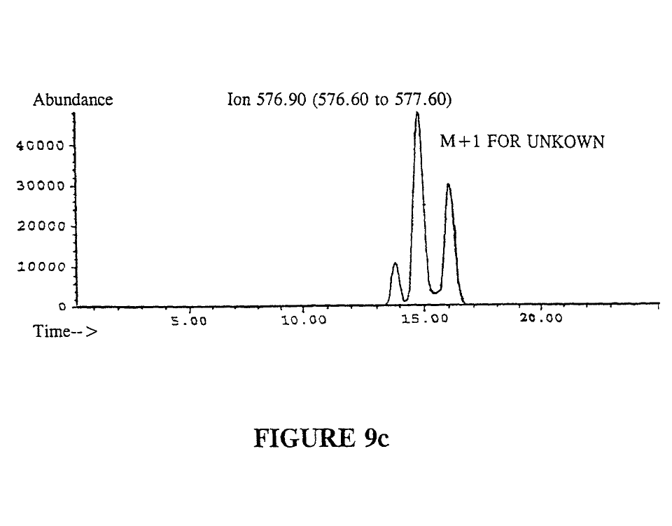

The upper and lower bands and band C were subjected to HPLC/MS, from which it was apparent that both upper and lower bands contained epicatechin and catechin (ion m/z 289), but also contained significant amounts of caffeine, whereas Band C contained predominantly caffeine. Furthermore, when electrospray MS was used, significant quantities of dimers of catechins (ion m/z 575) were detected in both the upper and lower bands, the proportions of the catechins and epicatechins, and their dimers appearing to differ between the upper and lower bands. FIGS. 7a and 7b show the quantities of catechin monomer (m/z 289) and dimer (m/z 575) detected by negative electrospray in the lower band, whilst FIGS. 8a and 8b show the quantities of catechin monomer and dimer respectively detected by negative electrospray in the upper band. FIGS. 9a, 9b and 9c illustrate respectively the quantities of catechin, sodium salt of catechin, and catechin dimer in the upper band as detected by positive electrospray. The quantities of catechin and its dimer detected in the lower and upper bands respectively, as determined from the areas under the peaks shown in FIGS. 7a, 7b, 8a and 8b are given in Tables 2 and 3 below.

TABLE 2
Determination of Relative Quantities of Catechin and Catechin Dimer in
HPLC Fractions of the Lower Band From the Areas Under The Peaks In
FIGS. 7a and 7b - Total Ion Chromatogram

| Retention Time (minutes) | Area | Area % | Ratio % |
|---|---|---|---|
| 11,280 | 9547267 | 37.940 | 79.754 |
| 12,699 | 11970864 | 47.571 | 100.000 |
| 13.290 | 3188541 | 12.671 | 26.636 |
| 14.660 | 457553 | 1.818 | 3.822 |

TABLE 3
Determination of Relative Quantities of Catechin and Catechin Dimer in
HPLC Fractions of the Upper Band From the Areas Under The Peaks In
FIGS. 8a and 8b - Total Ion Chromatogram

| Retention Time (minutes) | Area | Area % | Ratio % |
|---|---|---|---|
| 11.262 | 51957362 | 25.090 | 66.486 |
| 12.687 | 78148364 | 37.737 | 100.00 |
| 13.784 | 8729293 | 4.215 | 11.170 |
| 14.743 | 41863289 | 20.215 | 53.569 |
| 16.000 | 26388141 | 12.743 | 33.767 |

Band C was found to contain mainly caffeine, but smaller amounts of unidentified material) were also detected. Repeat analyses of the guarana fractions confirmed the above findings, but the amount of caffeine detected in band #8 was found to be variable.

On the basis of the fractionation experiments, and the platelet aggregation studies carried out on the fractions, it was concluded that the active fraction of guarana comprises a mixture of caffeine and catechins and/or epicatechins and their dimers.

### Example 2

Preparation of Caffeine-Epicatechin/Catechin Mixtures

Aqueous solutions (1 mM) of mixtures of catechin/epicatechin (Catechin—Sigma catalogue number C1251, Epicatechin—Sigma catalogue number E1753) and caffeine were made up in the ratios of catechin/epicatechin:caffeine of 1:2, 1:1 and 2:1 respectively along with 1 mM solutions of catechin/epicatechin or caffeine alone. Each of the solutions was then tested in the ADP induced platelet aggregation test described below. The platelet aggregation curves obtained (not shown) demonstrated that a 1:1 mixture of catechin and caffeine was a more effective inhibitor than either component alone.

It is not clear from these experiments whether the caffeine and catechins form a complex and the increased activity is due to the complex, although it is known that caffeine does form complexes with a number of compounds, see for example Reuning et al. J. Pharm. Sci. 1968; 57: 1341-1355.

Platelet Aggregation Studies

Platelet-rich plasma (PRP) was obtained by centrifuging blood collected in 3.8% trisodium citrate (9:1, v/v) from human volunteers or from the blood bank for 15 minutes at 700 rpm. Remaining blood was centrifuged at 2500 rpm for 25 minutes to obtain platelet-poor plasma (PPP). The platelets were quantified with an electronic counter (Coulter Electronics, Inc., Hialeah, Fla.). The platelet count was adjusted to 300,000-350,000/mm³ with PPP, and 0.45 ml used in each test of aggregation.

ADP (Sigma Chemical Co. St. Louis, Mo.) (0.1 mg/ml), and arachidonic acid (NuCheck Pre, Elysian, Minn.) (1.0 mg/ml in 20 mM Na₂CO₃) were used as aggregatory agents. Samples were warmed to 37° C. with stirring for approximately two minutes prior to the addition of 50 μl of aggregating agents. Guarana (or its fractions) or caffeine (alone or in combination with catechins) were added before the addition of ADP or arachidonic acid. The change in percentage light transmission was recorded with a dual aggregometer (Chrono-Log Co, Havertown, Pa.) after addition of aggregation agents.

### Example 3

Measurement of Thromboxane B₂ Synthesis

Rabbit platelet-rich plasma was centrifuged at 1000 g for 20 minutes. The resulting platelet pellet was washed twice in Tris-saline-glucose (TSG) buffer (15 mmol Tris. HD1; 134 mmol NaCl; 5 mmol d-glucose) containing 1 mM EDTA pH 7.4, according to the method described by Skjaerlund et al Biochemical Medicine 1983;30: 357-362. After the second washing, the platelets were suspended in phosphate-buffered saline (137 nM NaCl; 2.7 mM KCl; 0.9 mM CaCl₂; 0.8 mM MgCl₂, 2H₂O; 6.5 mM Na₂HPO₄. 2H₂O; 1.5 mM KH₂PO₄; 5.6 mM glucose; pH 7.4).

Prostaglandins generated by platelets were examined according to the methods described by Gerrard et al J. Lab. Clin. Med. 1980; 95: 950-958 and Wey et al Thrombosis and Haemostasis; 1982; 48: 94-97 with some modifications. Guarana extract or its fractions obtained by TLC were added to 0.5 ml platelet suspension in 20 μl volume. After two minutes preincubation at 37° C. in a shaking-bath, 0.1 μCi of [¹⁴C]-arachidonic acid (New England Nuclear, Boston, Mass.; sp. act. 55.8 Ci/mol) was added in a 100 μl volume of 10 mmol Na₂CO₃ (10). After ten minutes incubation in a shaking-bath at 37° C. the reaction was stopped by adding 0.5 ml 2M citric acid and 10 ml chloroform:methanol (2:1, v/v). The lower organic layer was collected following the addition of 1.0 ml 0.9% NaCl. The extract was evaporated to a small volume under N₂ followed by the addition of authentic standards for PGE₂ and TxB₂ carriers. The various prostaglandins were separated by TLC (250 μm silica gel G plates) using a solvent system consisting of diethylether; methanol; acetic acid (135:5:3, v/v/v). Prior to the separation, pure standards of PGE₂ and TxB₂ were spotted along the side samples to allow visualization following a brief exposure to 1′, 7′-dichlorofluorescein (0.1% in ethanol) spray. The areas corresponding to prostaglandins were scraped into vials, 10 ml of Aquasol-2 (New England Nuclear) added, and quantified by liquid scintillation counter (Packard Tri Carb Counter) with automatic external standard for quench correction. The remaining areas of the gel were also scraped and counted to determine percentage of recovery (always greater than 75%). Results were expressed as dpm of incorporation/2×10⁵ platelets.

The upper, lower and Band C fractions obtained by further chromatographic separation of Band #8 described above in Example 1 were assayed for their thromboxane synthesis inhibitory activity and the results are shown in Table 4 below.

TABLE 4
EFFECT OF ACTIVE FRACTIONS OF GUARANA ON
PROSTAGLANDIN SYNTHESIS FROM ¹⁴C ARACHIDONIC ACID
IN WASHED HUMAN PLATELETS

| | THROMBOXANE B₂ | PGE₂ |
|---|---|---|
| FRACTION | (dpm/mg protein) | (dpm/mg protein) |
| Control | 13,534 ± 474 | 1,645 ± 498 |
| GUARANA FRACTIONS | | |
| Band C | 9,495 ± 738^{a} | 1,894 ± 462^{a} |
| Upper | 2,989 ± 534^{a} | 607 ± 82^{a} |
| Lower | 8,201 ± 298^{a} | 1,052 ± 331^{a} |

^{a}Significantly different from controls

As can be seen from the Table, each of the three fractions had significant thromboxane synthesis inhibitory activity, with the upper fraction being most active.

### Example 5

Pharmaceutical Tablet Formulation

A tablet formulation containing the active fraction Band #8 is prepared by removing the solvent from the fraction, mixing the dried fraction intimately with lactose, starch and magnesium stearate in a cone blender and then compressing using a conventional rotary tabletting press to give a tablet having the following composition:

| | |
|---|---|
| Dried Fraction band #8 | 300 mg |
| Lactose | 630 mg |
| Starch | 60 mg |
| Magnesium stearate | 10 mg |

The resulting tablet is coated with a conventional film coating.

1. A pharmaceutical composition comprising a xanthine, and a catechin or oligomer or polymer thereof corresponding substantially to the anti-platelet aggregating catechin compounds isolable from Paullinia cupana, together with a pharmaceutically acceptable carrier.

2. A pharmaceutical composition having platelet aggregation inhibitory activity and comprising a mixture of xanthine and a polyphenol; and a pharmaceutically acceptable carrier.

3. A pharmaceutical composition comprising a mixture having synergistic platelet aggregation inhibitory activity, the mixture consisting essentially of a xanthine and a catechin or oligomers or polymers thereof; together with a pharmaceutically acceptable carrier.

4. A pharmaceutical composition comprising a mixture of a xanthine and a catechin or oligomer or polymer thereof, and a pharmaceutically acceptable carrier.

5. A pharmaceutical composition comprising a mixture of xanthine and a catechin or oligomer or polymer thereof, and a pharmaceutically acceptable carrier, the mixture being substantially free of polyphenols other than catechin and/or its oligomers and polymers.

6. A pharmaceutical composition according to any one of the preceding claims wherein the xanthine has the formula (I): 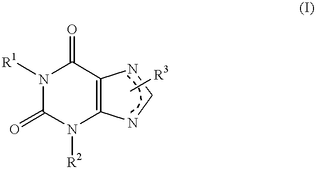
wherein R¹ is H or C₁₋₄ alkyl, R² is C₁₋₄ alkyl and R³ is bonded to either the 7 or 9 positions of the imidazole ring and in hydrogen or C₁₋₄ alkyl.

7. A composition according to claim 6 wherein R¹ is hydrogen or methyl, R² is methyl and R³ is hydrogen or methyl bonded to the 7-position of the imidazole ring.

8. A composition according to claim 7 wherein the xanthine derivative is selected from caffeine (R¹=R²=CH₃, R³=7-CH₃), theobromine (R¹=H, R²=CH₃, R³=7-CH₃) and theophylline (R¹=CH₃, R²=CH₃, R³=7-H) and mixtures thereof.

9. A composition according to claim 8 wherein the xanthine is caffeine.

10. A composition according to any one of the preceding claims wherein the xanthine and catechin are present in a molar ratio of between 1:5 and 5:1.

11. A composition according to claim 10 wherein the molar ratio of xanthine to catechin 1:2 to 2:1.

12. A composition according to claim 11 wherein the ratio is approximately 1:1.

13. A composition according to any one of the preceding claims wherein the catechin forms part of a purified fraction obtained from Paullinia cupana.

14. A pharmaceutical composition comprising a pharmaceutically acceptable carrier and a fraction isolable from P. cupana by extraction with ethanol, the fraction being characterised in that it:
(i) has platelet aggregation inhibition activity against ADP-induced platelet aggregation;
(ii) gives a positive colour test with vanillin/sulphuric acid reagent and concentrated hydrochloric acid reagent;
(iii) contains caffeine in complexed or uncomplexed form;
(iv) contains a catechin and/or an oligomer or polymer thereof; and
(v) has an Rf value between about 0.6 and about 0.8 (preferably between about 0.65 and about 0.75) when subjected to thin layer chromatography on 250 micron silica gel G eluted with chloroform: methanol: water (60:40:10 v/v/v).

15. A composition according to any one of the preceding claims which has platelet deaggregatory activity.

16. A pharmaceutical composition according to any one of the preceding claims which is in the form of a tablet, capsule, lozenge or granules.

17. A pharmaceutical composition according to anyone of the preceding claims which is in unit dose form.

18. A pharmaceutical composition according to claim 17 wherein the unit dose contains from 50 mg to 500 mg of a mixture of the xanthine and the polyphenol.

19. A pharmaceutical composition according to claim 18 wherein the unit dose contains from 150 mg to 350 mg of a mixture of the xanthine and the polyphenol.

20. The use of a catechin or oligomers or polymers thereof, and a xanthine, as defined in any one of the preceding claims, for the manufacture of a medicament for the treatment of platelet aggregation mediated conditions.

21. The use of a combination of a xanthine and a polyphenol as defined in any one of the preceding claims, for the manufacture of a medicament for the treatment, alleviation or prevention of diseases or conditions mediated by thromboxanes such as thromboxane B2

22. A method of treatment of a disease or condition mediated by platelet aggregation in a patient (e.g. a mammalian patient such as a human) in need of such treatment, which method comprises administering to the patient a non-toxic therapeutically effective amount of a composition as defined in any one of the preceding claims.

23. A method of treatment of a disease or condition mediated by thromboxanes in a patient (e.g. a mammalian patient such as a human) in need of such treatment, which method comprises administering to the patient a non-toxic therapeutically effective amount of a composition as defined in any one of the preceding claims.
